(19) United States
(12) Patent Application Publication
(10) Pub. No.: US 2022/0370181 A1
(43) Pub. Date: Nov. 24, 2022

(54) SYSTEMS AND METHODS FOR MONITORING AND REGULATING BLADDER FUNCTION AND MECHANICS

(71) Applicant: VESI CORPORATION, Leander, TX (US)

(72) Inventors: Samuel A. WEPRIN, Richmond, VA (US); Zachary CULLINGSWORTH, Richmond, VA (US); John A. NOEL, Philadelphia, PA (US); John E. SPEICH, Henrico, VA (US); Wesley BOSMAN, Richmond, VA (US); Jordon C. FRIEDMAN, Brooklyn, NY (US); Adam P. KLAUSNER, Midlothian, VA (US); Jesse D. ADAMS, Mars, PA (US)

(73) Assignee: VESI CORPORATION, Leander, TX (US)

(21) Appl. No.: 17/817,071

(22) Filed: Aug. 3, 2022

Related U.S. Application Data
(63) Continuation of application No. PCT/US2021/016459, filed on Feb. 3, 2021.
(60) Provisional application No. 62/969,210, filed on Feb. 3, 2020.

(51) Int. Cl. A61F 2/00; A61B 17/135

(52) U.S. Cl. CPC A61F 2/0031; A61F 2/0009; A61B 17/1355; A61B 2017/00199

(57) ABSTRACT

Devices, methods, and systems for controlling a bladder of a subject are disclosed. For example, an apparatus for controlling the bladder can comprise a garment or accessory configured to be worn at least partially around a lower truncal region of a subject and a compression device coupled to the garment or accessory. The compression device can be configured to pressurize an abdominal wall of the subject when the garment or accessory is worn by the subject. The compression device can comprise a device housing having a contact surface, a device base, and an actuator. The actuator can translate the device housing in relation to the device base in a medial or lateral direction relative to the subject such that the contact surface of the device housing applies compressive forces to the abdominal wall of the subject when translated.

### CROSS-REFERENCE TO RELATED APPLICATIONS

This application is a continuation of International Patent Application No. PCT/US2021/016459 filed on Feb. 3, 2021, which claims the benefit of priority of U.S. Provisional Application No. 62/969,210 filed on Feb. 3, 2020, the contents of which are incorporated herein by reference in their entities.

### TECHNICAL FIELD

The present disclosure relates generally to the field of urological medical devices and methods and, more specifically, to devices and methods for training and controlling the bladder of a subject.

### BACKGROUND

Overactive Bladder (OAB) is a common condition that affects as many as 1 in 3 adults over the age of 40. OAB is the result of the bladder contracting at a higher than normal rate, even during periods of lower volumes of urine in the bladder. Common symptoms include incontinence and urgency and frequency of urination, impairing quality of life of those affected.

Current methods of OAB therapy include behavioral modifications (e.g., decreasing fluid intake, timed voiding, avoiding bladder irritates), pelvic floor therapy (e.g., pelvic muscle exercises, tibial nerve simulators), medications that block receptors to the bladder, intravesical treatments, and surgery (e.g., bladder augmentation, diversion). However, the current methods of OAB therapy have various disadvantages or shortcomings. For example, behavioral modifications, pelvic floor therapy, and medications often have poor compliance. Over 50% of patients stop taking oral therapies within several months of starting such therapy. Further, pelvic floor muscle training is often recommended but is not directed to the bladder nor can it aid with training the bladder.

Therefore, devices and methods are needed to enable a patient's bladder to allow larger volumes of urine at any given pressure. There also exists a need for such devices and methods to be non-invasive, discreetly wearable, and user-friendly, allowing patients more compliance with therapy and control over their OAB symptoms throughout the day.

### SUMMARY

Devices, methods, and systems for controlling a bladder of a subject are disclosed. For example, an apparatus for controlling the bladder can comprise a garment or accessory configured to be worn at least partially around a lower truncal region of a subject and a compression device coupled to the garment or accessory. The compression device can be coupled to the garment or accessory. The compression device can be configured to pressurize an abdominal wall of the subject when the garment or accessory is worn by the subject.

The garment can be a pair of compression shorts or the like. The accessory can be a belt or the like. The garment can also be a pair of compression shorts comprising a belt.

The compression device can comprise a device housing. The device housing can comprise a contact surface. The compression device can also comprise a device base. The compression device can also comprise a threaded shaft. The threaded shaft can couple the device base to the device housing. The compression device can also comprise an actuator. The actuator can be configured to translate the device housing in relation to the device base in a medial or lateral direction relative to the subject such that the contact surface of the device housing applies compressive forces to the abdominal wall of the subject when translated. The compression device can also comprise a portable power supply (e.g., a battery) configured to power the actuator.

The actuator can comprise a shape memory actuator. The shape memory actuator can be configured to translate the device housing in relation to the device base in response to a temperature change. The bladder control apparatus can further comprise a heating element. The heating element can be configured to apply heat to the shape memory actuator to change a shape of the shape memory actuator. The shape memory actuator can comprise one or more shape memory flaps. The shape memory flaps can be configured to translate the device housing in relation to the device base in response to the temperature change. The shape memory actuator can also comprise one or more shape memory springs. The shape memory springs can be configured to translate the device housing in relation to the device base in response to a temperature change. The one or more shape memory springs can be configured to change the shape of a deformable sheet. The deformable sheet can make up part of the device housing. The shape memory actuator can be made in part of at least one of a shape memory alloy and a shape memory polymer.

The actuator can further comprise a threaded shaft (e.g., leadscrew or part of a worm gear). The threaded shaft can couple the device base to the device housing. The actuator can also comprise a rotary gear. The rotary gear can be configured to rotate the threaded shaft to translate the device housing in relation to the device base. The compression device can further comprise a heating element. The heating element can be configured to apply heat to the abdominal wall of the subject.

The compression device can also be configured to be in wireless communication with a remote device. The compression device can be configured to apply compressive forces to the abdominal wall of the subject automatically in response to a user input applied to the remote device. The user input can be a manipulation of a sensation meter graphic. The sensation meter graphic can be shown on a display of the remote device. The compression device can further comprise an ultrasound unit comprising an ultrasound probe. The ultrasound unit can be configured to detect a volume within the bladder of the subject. The compression device can be communicatively coupled to the ultrasound unit. The compression device can be configured to automatically apply compressive forces to the abdominal wall of the subject in response to the volume detected by the ultrasound unit.

The contact surface of the device housing can be substantially planar.

The apparatus can further comprise a fluid-filled covering. The fluid-filled covering can be configured to cover at least part of the device housing. The fluid-filled covering can be configured to conform to an anatomy of the subject. The fluid-filled covering can be filled with air or another type of gas. The fluid-filled covering can also be filled with a liquid. The fluid-filled covering can also be filled with particulates. In this variation, the fluid-filled covering can also apply force(s) to the abdominal wall via a pump (e.g., a fluid pump, a pneumatic based pump, or the like).

One variation of a method of controlling a bladder of the subject can comprise securing a compression device to the subject. The compression device can be secured to the subject using a garment or accessory. The garment or accessory can be configured to be worn at least partially around a lower truncal region of a subject. The apparatus can comprise a compression device. The compression device can be coupled to the garment or accessory. The compression device can be configured to pressurize an abdominal wall of the subject when the garment or accessory is worn by the subject.

The compression device can comprise a device housing. The device housing can comprise a contact surface. The compression device can also comprise a device base. The compression device can also comprise a threaded shaft (e.g., leadscrew or part of a worm gear). The threaded shaft can couple the device base to the device housing. The compression device can comprise an actuator. The actuator can be configured to translate the device housing in relation to the device base in a medial or lateral direction relative to the subject such that the contact surface of the device housing applies compressive forces to the abdominal wall of the subject when translated. The compression device can further comprise a portable power supply (e.g., a battery) configured to power the actuator. The method can comprise translating the device housing in relation to the device base such that the contact surface of the device housing applies compressive forces to the abdominal wall of the subject when translated.

Also disclosed is a bladder control system. The bladder control system for controlling the bladder can comprise a garment or accessory. The garment or accessory can be configured to be worn at least partially around a lower truncal region of a subject. The system can further comprise a compression device. The compression device can be coupled to the garment or accessory. The compression device can be configured to pressurize an abdominal wall of the subject when the garment or accessory is worn by the subject.

The compression device can comprise a device housing. The device housing can comprise a contact surface. The compression device can also comprise a device base. The compression device can further comprise a threaded shaft. The threaded shaft can couple the device base to the device housing. The compression device can further comprise an actuator. The actuator can be configured to translate the device housing in relation to the device base in a medial or lateral direction relative to the subject such that the contact surface of the device housing applies compressive forces to the abdominal wall of the subject when translated. The compression device can also comprise a portable power supply (e.g., a battery) configured to power the actuator. The system can further comprise a remote device in wireless communication with the compression device. The remote device can be configured to provide feedback to the compression device in response to a characteristic of the bladder of the subject.

### BRIEF DESCRIPTION OF THE DRAWINGS

FIG. 1A illustrates a variation of the compression device in a resting configuration.

FIG. 1B illustrates a variation of the compression device in an expanded configuration.

FIG. 1C illustrates a device base of a variation of the compression device.

FIG. 1D illustrates a threaded shaft of a variation of the compression device.

FIG. 1E illustrates a variation of the compression device with a belt and elastic bands.

FIG. 1F illustrates a variation of the compression device wrapped along the lower truncal region of a subject.

FIG. 2 illustrates another variation of the compression device having a circular contact surface.

FIG. 3A illustrates a variation of a shape-memory flap actuator having flaps before activation.

FIG. 3B illustrates a variation of the shape-memory flap actuator having flaps after activation.

FIG. 4A illustrates a variation of a spring actuator having a shape memory spring in a compressed configuration.

FIG. 4B illustrates a variation of the spring actuator having a shape memory spring in an extended configuration.

FIG. 5A illustrates a variation of a bendable shape memory actuator in a non-bent state.

FIG. 5B illustrates a variation of the bendable shape memory actuator in a bent state.

FIG. 6 illustrates a garment having a variation of the compression device coupled thereto.

FIG. 7 illustrates a drawing of communication and power systems within a variation of the compression device.

FIG. 8 illustrates a drawing of database systems communicating with the compression device.

FIGS. 9A-9D illustrate various graphical user interfaces provided through a display of the remote device.

FIGS. 10A-10D illustrate various displays of a variation of a doctor dashboard.

FIG. 11A illustrates a manual feedback loop representing an example method of operation of the compression device and the remote device.

FIG. 11B illustrates an automatic feedback loop representing another example method of operation of the compression device and the remote device.

FIG. 12 illustrates a garment having a variation of the compression device and a battery coupled thereto.

FIGS. 13A-13C illustrate variations of belts having different tensioning mechanisms.

### DETAILED DESCRIPTION

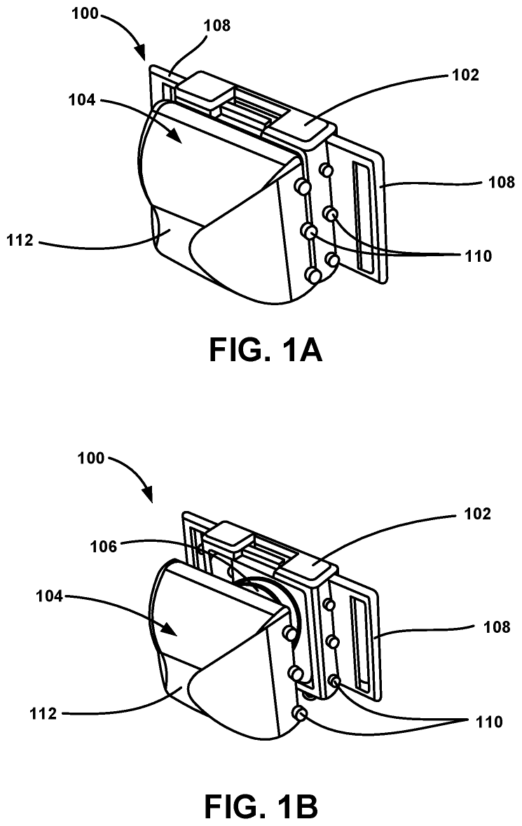

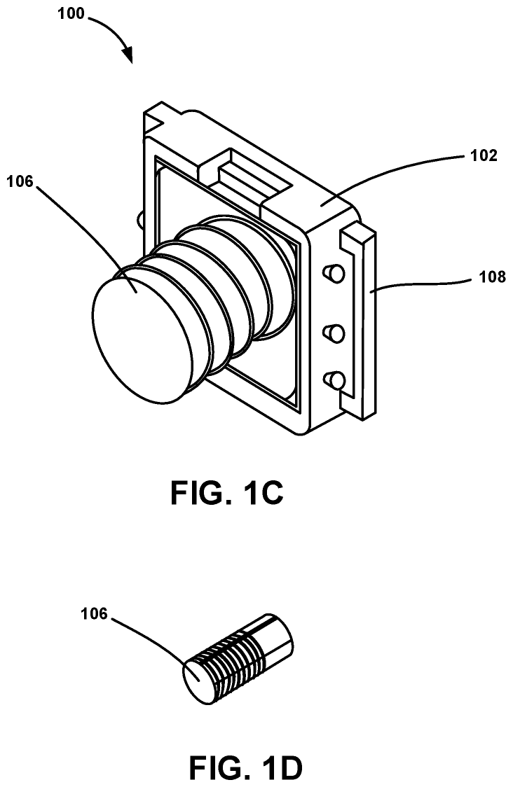

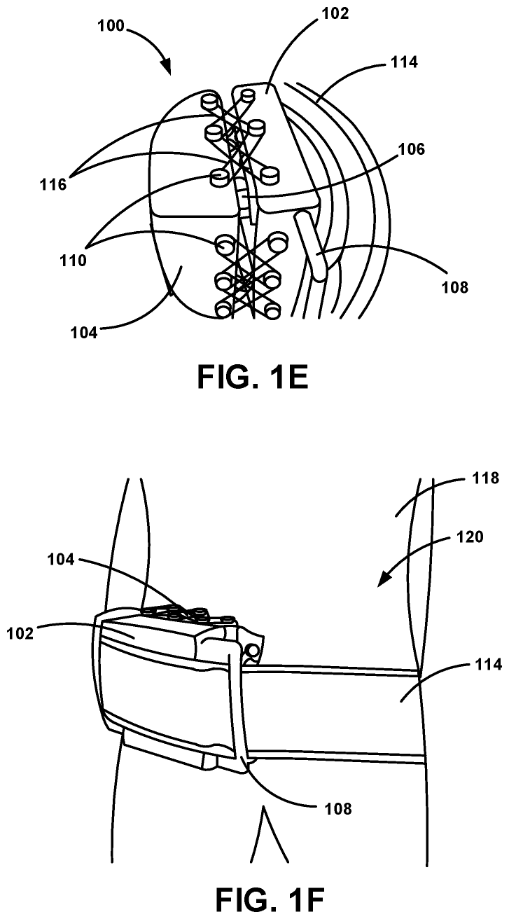

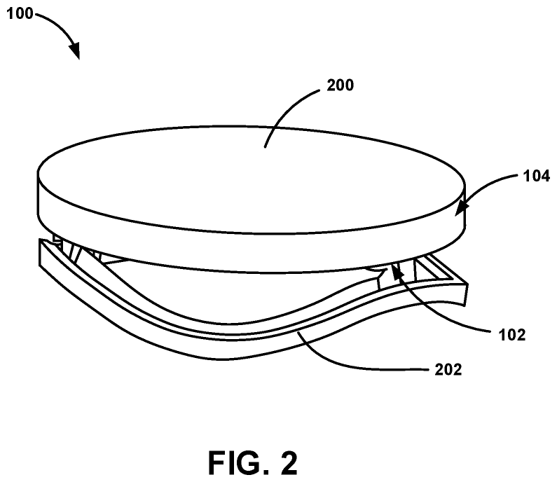

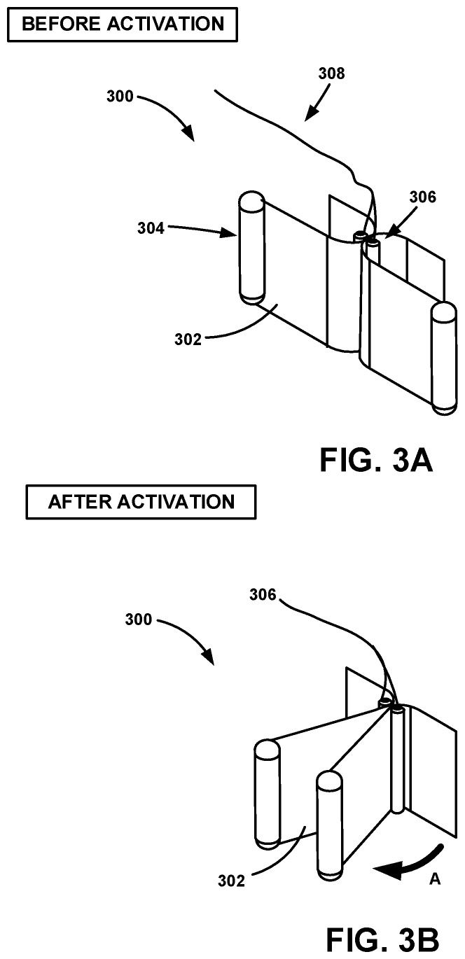

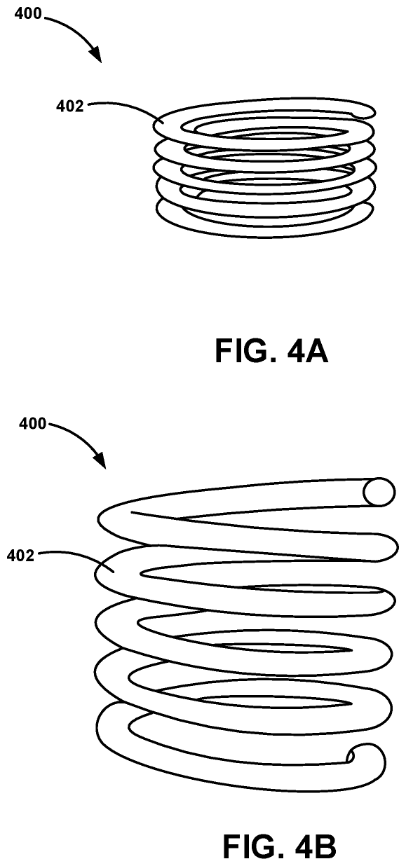

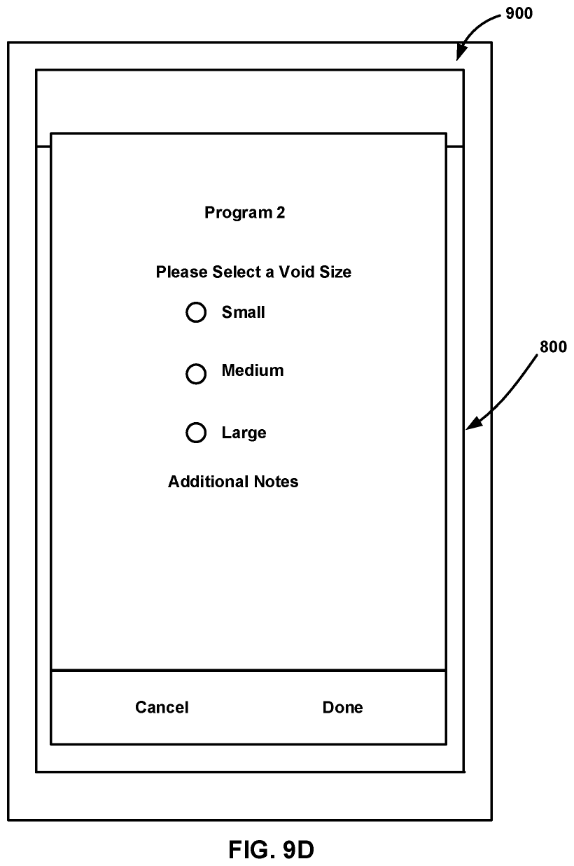

FIGS. 1A and 1B illustrate variations of the compression device 100 in first and second configurations. The compression device 100 can comprise a device base 102 and a device housing 104 configured to move relative to the device base 102. The compression device 100 can be configured to pressurize an abdominal wall of the subject positioned near the subject. The compression device 100 can further comprise a threaded shaft 106, a belt securement loop 108, and nodular posts 110. One variation of compression device 100 for controlling the bladder can comprise a garment or accessory (see, e.g., belt 114 in FIGS. 1F, 2, and 13A-13C and garment 600 in FIG. 6). The compression device 100 can be coupled to the garment or accessory via the belt securement loop 108. This mechanism can secure the compression device 100 to the subject at a region where the compression device 100 is positioned in proximity to the bladder. For example, the garment or accessory can be configured to be worn at least partially around a lower truncal region of a subject.

The device base 102 and the device housing 104 can be connected via the threaded shaft 106 and can be substantially parallel to each other. In the first configuration, the device housing 104 can be positioned in a resting configuration, as seen in FIG. 1A. When positioned adjacent to the subject's abdominal wall, device housing 104 can move back and forth with respect to the device base 102, contacting the abdominal wall of the subject and delivering compressive forces to the abdominal wall in proximity to the bladder of the subject. As such, the device housing 104 can be made of a material that can safely and flexibly contact the body of the subject, such as soft silicone, plastic, or the like. The device base 102 and other structural components can remain in place during compression and can be made of materials such as rigid plastic, aluminum, metal, or the like.

The compressive forces can induce stress softening (strain softening) and thus result in improved dynamic elasticity within the bladder of the subject. Since symptoms of frequency and urgency of urination occur at lower volumes within the bladder, strain via external compression can train the detrusor muscle within the bladder. This training can regulate bladder elasticity and thus can effectively increase the bladder's ability to hold more volume at any given pressure.

FIG. 1B illustrates a variation of the compression device 100 in an expanded configuration. As such, the device housing 104 can comprise a contact surface 112 that contacts the abdominal wall to apply or relieve compressive forces to the abdominal wall in the second configuration. The device housing 104 can also rotate circumferentially during use. The contact surface 112 can be beveled or chamfered to enhance comfortability for the subject. The contact surface 112 can also be substantially planar. This can allow a subject to wear the device discreetly. The device housing 104 can also have a substantially flat base.

FIG. 1C illustrates a device base 102 of the compression device 100 separated from the device housing 104 (i.e., the device housing 104 is removed). As seen in FIG. 1C, the threaded shaft 106 can extend towards the device housing 104 in a perpendicular direction to a planar surface of the device base 102. As such, the resulting compressive forces can be directed at the abdomen of the subject in a radially inward direction or in a medial direction when the compression device 100 is activated and coupled to the garment/accessory worn by the subject.

The threaded shaft 106 can be attached to the device base and fit into a recess of the device housing 104. The threaded shaft 106 can couple the device base 102 to the device housing 104 and can be configured to allow the device housing 104 to move in relation to the device base 102 in order to compress the abdominal wall of a subject. Alternatively, the threaded shaft 106 can be attached to the device housing 104 and fit into a recess of the device base 102. The threaded shaft 106 can be a lead screw, a worm gear, or any other suitable mechanisms for translating the device housing 104 with respect to the device base 102. A variation with a lead screw can have a motor and drive pulley coupled to the motor and configured to power the rotation of the lead screw. A variation with a worm gear can comprise a worm wheel and a worm barrel configured to rotate with respect to the device housing 104.

Additionally, the compression device 100 can comprise a stepper motor (not shown) configured to rotate the threaded shaft 106 to oscillate the device housing 104 in relation to the device base 102. The stepper motor can be attached directly to a rotary gear, adding durability to the device 100. The stepper motor can be placed within the threaded shaft 106, within the device base 102 or device housing 104, or another location within the device 100. The stepper motor can be sized to achieve precise positioning and control the translational speed of the device housing 104 by controlling the rotation of the threaded shaft 106. The stepper motor can divide a full rotation into an equal number of loops, allowing a motor controller to command the rate and magnitude of the compressive forces placed on the abdominal wall. In one example, the stepper motor can be a high-torque NEMA 23 or NEMA 24 bipolar stepper motor having a step angle of approximately 1.8 degrees and a holding torque of between about 2.40 Newton-meter (N-m) and 3.5 N-m.

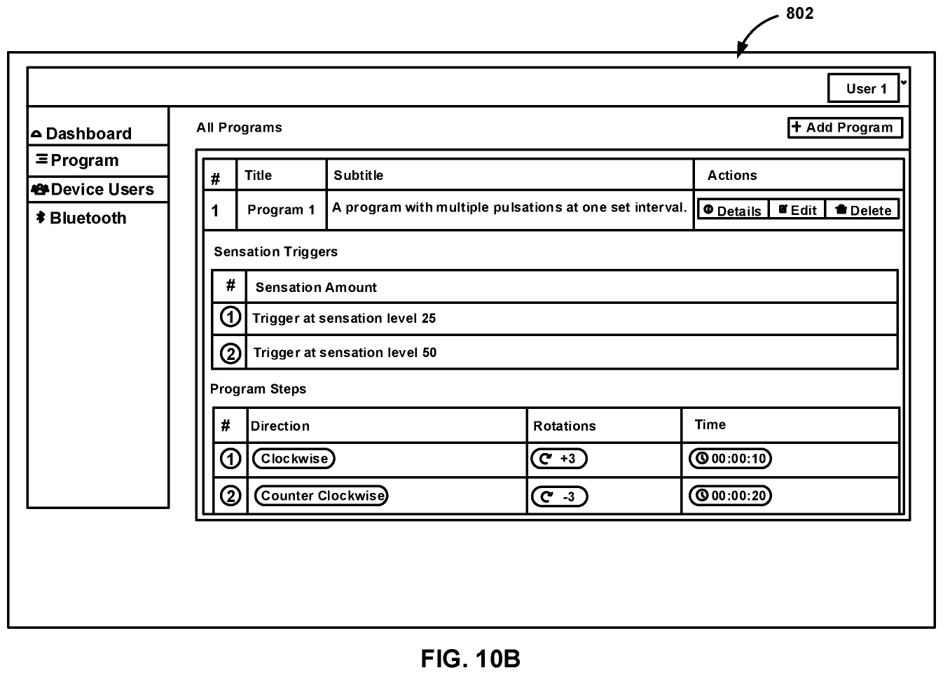

To perform designed bladder compressions, the rotary gear and the stepper motor can be activated to rotate the threaded shaft 106 to translate the device housing 104 in relation to the device base 102. Oscillations and compression forces can be customized according to different subject needs. For example, a magnitude or a rate of compression can be adjusted or set by the subject or another user. The device housing 104 can also rotate clockwise or counterclockwise in relation to the subject's abdominal wall. Such control may be executed via an attached or remote device 800 (see FIG. 8).

FIG. 1D illustrates another variation of the threaded shaft 106. As shown in FIG. 1D, the dimensions of the threaded shaft 106 can vary (e.g., the size of the threaded shaft 106 can be reduced or miniaturized) depending on the desired dimensions of the compression device 100.

FIG. 1E illustrates a variation of the compression device 100 with a belt 114 and elastic bands 116 detachably coupling the device base 102 to the device housing 104. The compression device 100 can have a belt 114 passing through the belt securement loop 108. The belt securement loop 108 can be located on one or both lateral sides of device base 102. Accordingly, the belt securement loop 108 can hold a belt 114 that can wrap around the subject. The belt 114 can be configured such that it is built into a clothing garment (e.g., stretch shorts, leggings, pants, undergarments). Alternatively, the belt 114 can be worn as a standard belt (i.e., through loops of a clothing garment). The belt 114 can be Velcro-based and can be made of any suitable material, including nylon. The belt 114 can be inflatable to allow for elasticity of the entire device 100 during repeated compression cycles. In yet another variation, the compression device 100 can be worn without a belt 114, (i.e., held within the clothing of the subject).

The device housing 104 and the device base 102 can comprise one or more nodular posts 110 located on outer surfaces of the device housing 104 and the device base 102. The nodular posts 110 can be circular protrusions, though it should be considered that various protrusions shapes can be considered.

The base and the housing can be coupled. One method of coupling can include the use of elastic bands 116 (e.g., rubber bands or other types of elastic bands) can loop around the nodular posts 110 to couple the device housing 104 to the device base 102 while the device base 102 is being moved in relation to the device housing 104. This can ensure that the device housing 104 and the device base 102 stay connected to each other in the first and second configurations, helping maintain discretion for the subject during daily activities. This can also help allow the user to maintain continuous exercise throughout the day, whether such exercise is for bladder therapy or otherwise.

FIG. 1F illustrates a variation of the compression device wrapped along the lower truncal region or lower abdomen 120 of a subject 118. While the belt 114 is coupled to the compression device 100 and wrapped around the subject, the compression device 100 can apply compressive forces to the abdominal wall. The belt 114 can assist the compression device 100 to maintain a position over the abdomen such that the subject 118 does not have to constantly adjust or monitor the position of the compression device 100, ensuring constant therapy as desired.

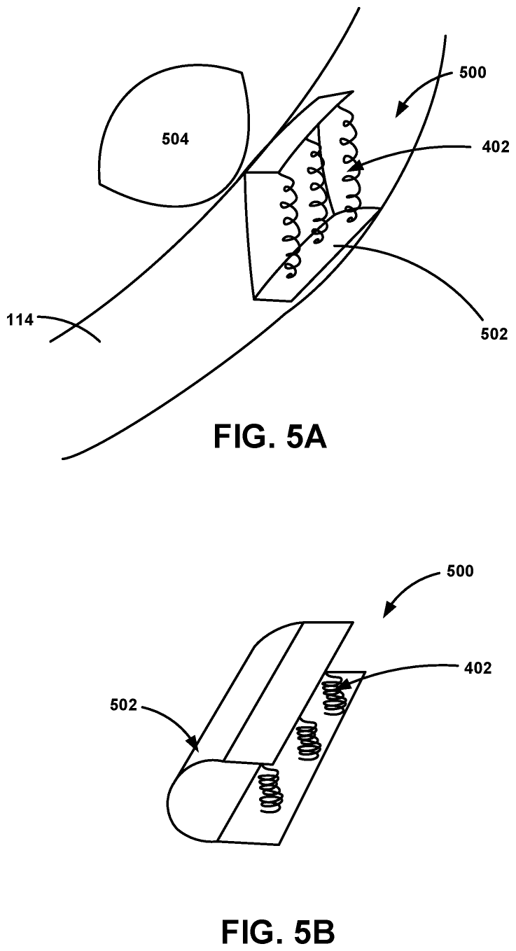

FIG. 2 illustrates another variation of the compression device having a circular planar contact surface 200. The planar contact surface 200 can be used in a similar manner as the compression device 100 illustrated in FIGS. 1A to 1F. As such, the motor within can be used to lift the planar contact surface 200 with respect to the device base 102. In this variation, the device base 102 can be configured to sit on a track 202. The track 202 can support the device base 102 and can attach to an underlying belt or garment for securement to the subject. It should be understood that the contact surface can be provided in various shapes and not necessarily be planar (e.g., beveled).

The compression device 100 can also comprise an actuator. The actuator can further comprise a threaded shaft 106 that can couple the device base 102 to the device housing 104. The actuator can be configured to translate the device housing 104 in relation to the device base 102 in a medial or lateral direction relative to the subject such that the contact surface 112 of the device housing 104 applies compressive forces to the abdominal wall of the subject when translated. The compression device 100 can also comprise a portable power supply configured to supply power to the actuator.

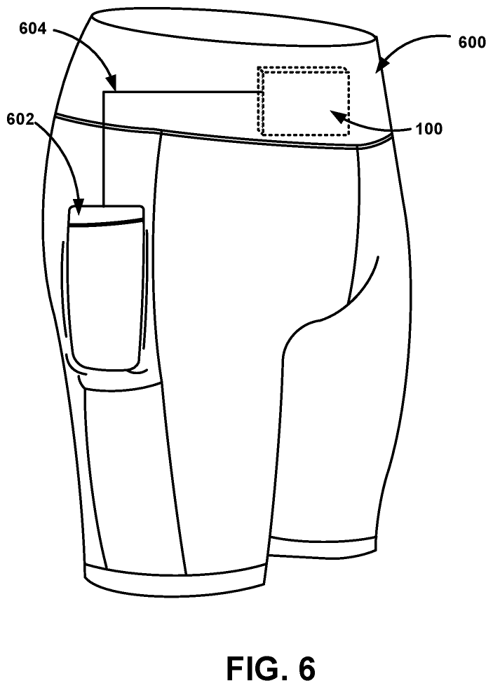

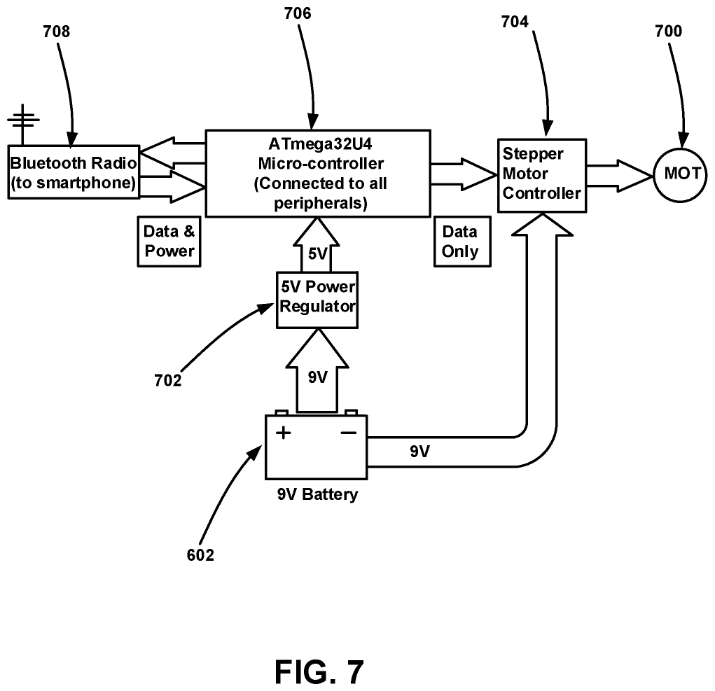

FIG. 3A illustrates a variation of a shape-memory flap actuator 300 having one or more shape-memory flaps 302 before activation. The shape-memory flaps 302 can be made of nitinol, or any other suitable shape-memory alloy or polymer. Before activation, the shape-memory flaps 302 can be configured parallel or substantially parallel to the contact surface 112 of device housing 104. A rounded edge 304 can border the shape-memory flaps 302 such that nearby elements of the compression device are undamaged.

The compression device 100 can further comprise a heating element 306. The heating element 306 can be provided within the compression device 100 to change the shape of the shape memory actuator, such as the shape-memory flaps 302. The heating element 306 can be connected to an external power source (not shown) via wires 308. The wires 308 can run through the hem of the garment or alongside the garment and secured in place to prevent the wire from twisting or coiling. The shape memory flaps 302 can be configured to translate the device housing 104 in relation to the device base 102 in response to the temperature change.

FIG. 3B illustrates a variation of the shape-memory flap actuator 300 after activation. Heating the shape-memory flaps 302 to a predetermined temperature using the heating element 306 can change the shape of the shape-memory flaps 302. The shape-memory flaps 302 can be translated in a predetermined direction, for example as seen by arrow A. The shape-memory flaps 302 can change shape at a temperature of 50° Celsius, though it is to be understood that the predetermined temperature can be at various temperatures. In other configurations, the shape changing element may be actively heated with the assistance of a current.

The temperature of the shape-memory flaps 302 can be altered using mechanisms of conduction and convection via the heating element 306. Conduction and convection can occur individually or in combination to achieve a desired temperature to produce a desired conformation of the shape-memory flaps 302. Heating and cooling by conduction can also allow for body heat to conduct from the body of the subject into the shape-memory flaps 302. Additionally, cooling by conduction can involve allowing heat to conduct out of the shape-memory flaps 302 into other surrounding parts of the compression device 100. As such, the shape-memory flaps 302 and other surrounding parts can be in contact with thermally conductive material to exhaust heat. Parts can also be affected by cooler air or surface external to the device to exhaust heat. Heating and cooling by convection can involve allowing warm and cool fluid, respectively, to pass over the shape-memory flaps 302. The cooling fluid can also be air or other fluids such as water.

After activation, the shape-memory flaps 302 change shape to project in a direction towards the device housing 104. This shape change can directly apply force and pressure onto the abdominal wall of the subject, compressing the abdominal wall. The shape-memory flaps 302 can act as a lever in this manner, producing force onto the abdominal wall. If desired, the shape-memory flaps 302 can be configured to deliver a varying level of force by varying the temperature applied thereto. Alternatively, an intermediary component such as a spring or pressure reservoir can be used to divert or store a portion of the force generated by the shape-memory flaps 302. The shape memory actuator can also be in the form of any mechanical structure that generates force. Further, the shape memory actuator can be configured so that contraction of the shape-memory actuator provides force to the abdominal wall.

The shape memory actuator can also comprise a spring actuator 400 comprised as one or more shape memory springs 402. Like the shape-memory flaps 302, the shape memory springs 402 can be configured to translate the device housing 104 in relation to the device base in response to a temperature change.

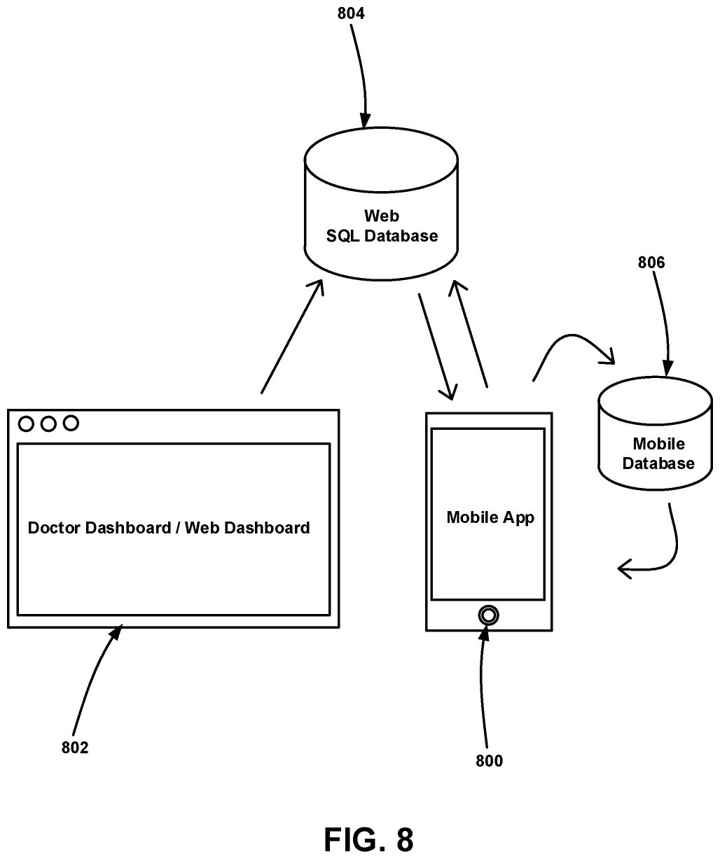

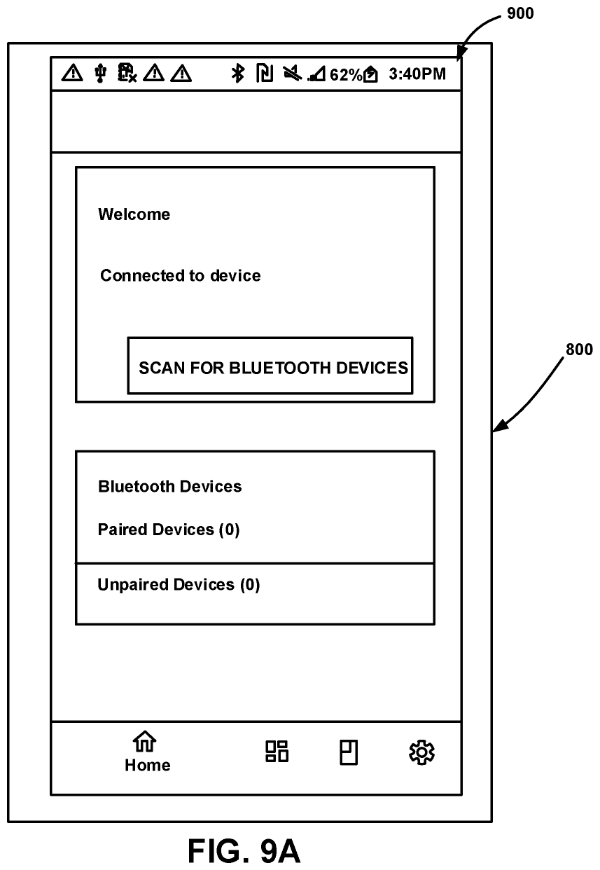

FIG. 4A illustrates a variation of a spring actuator 400 having a shape memory spring 402 in a compressed configuration. The shape memory spring 402 can have a width of 2 inches, a depth of 2 inches, and a cold height of 1 inch, though any variations of dimensions can be considered. Like the shape-memory flaps 302, the shape memory spring 402 can be made in part of nitinol, or any other suitable shape-memory alloy or polymer.

FIG. 4B illustrates a variation of the spring actuator having a shape memory spring 402 in an extended configuration. The extended length in the extended configuration can be 2 inches, though any length can be considered. Like the shape-memory flaps 302, the shape memory spring 402 can be configured to change shape at a desired temperature. Once the shape-memory spring 402, reaches a desired temperature, the shape-memory spring 402 can extend to provide pressure to the abdominal wall. Alternatively, the shape-memory spring 402 can be configured to provides force to the abdomen during contraction.

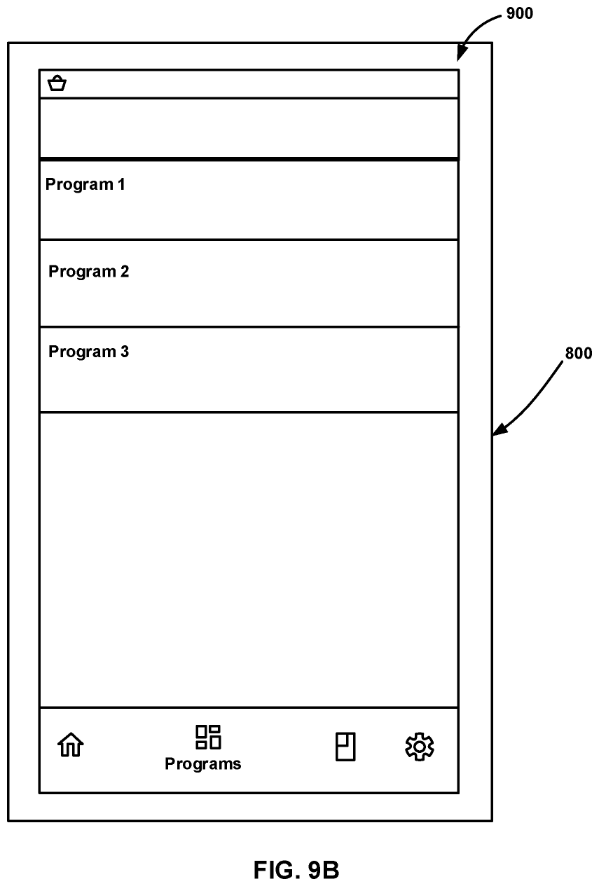

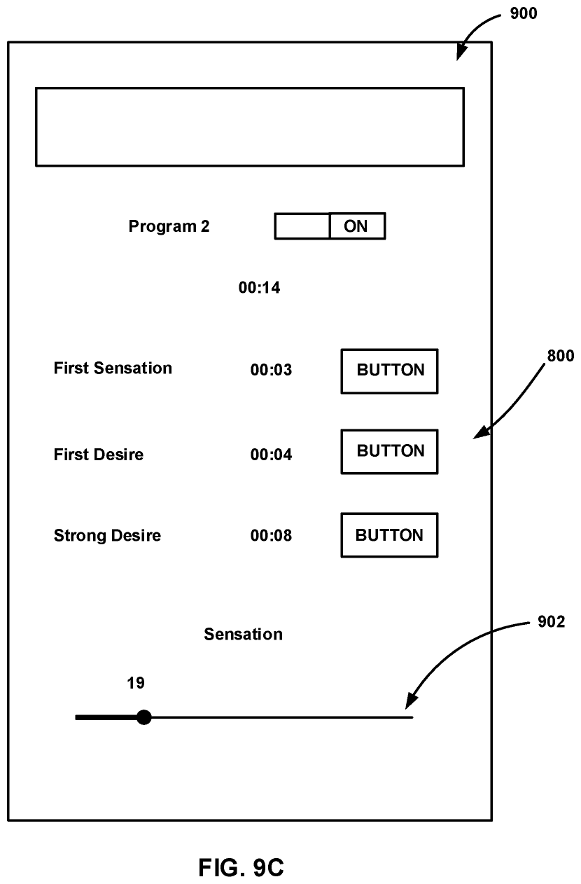

The actuator can also comprise another type of shape memory actuator 500 (see, e.g., FIGS. 5A and 5B). For example, the shape memory springs 402 can be configured to change the shape of a bendable shape memory sheet 502.

The shape memory actuator 500 can be configured to translate the device housing in relation to the device base in response to a temperature change. The shape memory actuator can comprise one or more shape memory flaps.

FIG. 5A illustrates a variation of a bendable shape memory actuator 500 in a non-bent state. This variation can comprise a bendable shape memory sheet 502, shape memory springs 402, and a belt 114 or garment band. The bendable shape memory sheet 502 can make up part of the device housing 104. The bendable shape memory sheet 502 can be attached to the belt 114 in order to contact the abdominal wall of the subject while the belt 114 worn. The shape memory springs 402 can be attached to the bendable shape memory sheet 502 and can be compressed via a heating element as described above.

FIG. 5B illustrates a variation of the bendable shape memory actuator in a bent state. When the shape memory springs 402 are compressed, the bendable shape memory sheet 502 bends or rolls. This bending or rolling causes the bendable shape memory sheet 502 to bulge towards the abdominal wall, providing force and compression to the bladder 504 of the subject. Alternatively, the heating element can change the temperature of the bendable shape memory sheet 502 directly, causing the shape memory springs 402 to bend in response to the bending of the bendable shape memory sheet 502. The heating element can also change the temperature of both the bendable shape memory sheet 502 and the shape memory springs 402 at the same time.

The heating element 306 within the compression device 100 can also deliver heat directly to the abdominal wall over the bladder. Heat can be delivered to the abdomen either separately or concurrently with force from device housing 104. The heating element 306 can be placed within the threaded shaft 106, within the device base 102, within the device housing 104, or another location within the compression device 100. In such configurations, the compression device 100 can have a thermal insulator to protect the subject 118. The thermal insulator can bring the compression device 100 to a suitable temperature for contact with the subject.

The compression device 100 can further comprise a fluid-filled covering. This mechanism can further assist in heating or cooling a shape-memory alloy, for example. Such a covering can alternatively be used as a membrane to protect the abdominal wall from overheating. As such, the fluid-filled covering can be configured to cover at least part of the device housing and can be configured to conform to an anatomy of the subject. Additionally, the fluid-filled covering can be filled with an air, a gas, or a liquid. Alternatively, the fluid-filled covering can also be filled with particulates.

An inflatable membrane can be provided with the compression device 100 to cover the compression device 100 and fit the anatomy of the subject. The inflatable membrane can be in the form of a drape, a pouch, or an inflatable wrap. The subject can manually inflate or deflate the inflatable membrane as desired. The inflatable membrane can either be attached to the compression device 100 or can be separate to the device 100 for the subject to attach themselves as desired.

An inflatable membrane can also serve as an independent method to apply compressive force to the abdomen through manual or automated inflation with or without an underlying base and may be secured to the belt or garment directly.

The compression device 100 can also comprise a vibration motor that applies vibration perpendicular to the abdominal wall overlying the bladder. Vibration can occur simultaneously with external compression or independently to the external compression.

FIG. 6 illustrates a garment 600 having a variation of the compression device 100 coupled thereto. The garment can be compression shorts as shown, but can also be any wearable garment such as leggings, pants, undergarments, or the like. The compression device 100 can be worn within the garment with or without belt 114. The shape of the compression device can be such that a subject can wear the device discreetly, allowing the subject peace of mind for daily activities. To power the compression device, a battery 602 can be provided to be held within the garment 600. The battery may be connected to the compression device 100 via a lead wire 604.

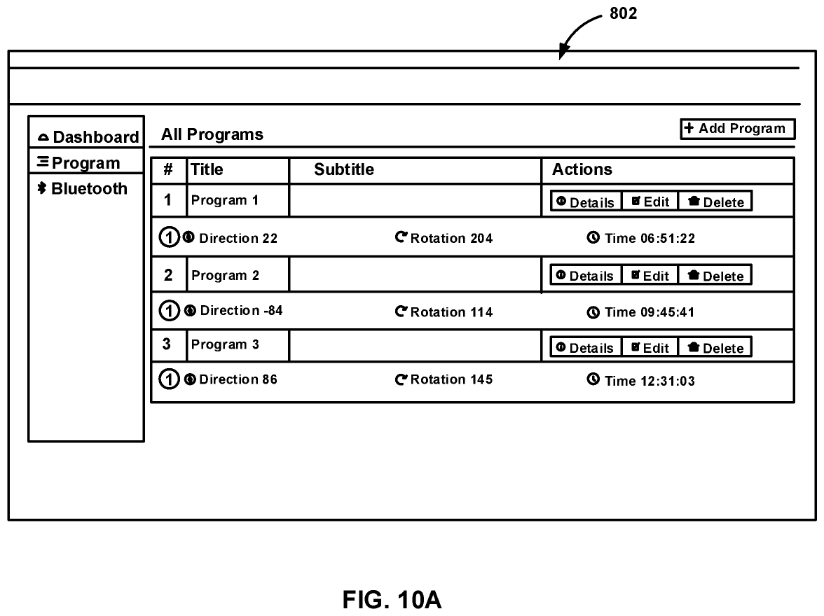

FIG. 7 illustrates a drawing of communication and power systems within a variation of the compression device 100. The electrical components within the belt can contribute to a small footprint as well as eliminating the need for any belt-side updates. A system within compression device 100 can run a motor 700, such as a stepper motor as previously described, resulting in movement of the device housing 104 with respect to the device base 102. The motor 700 can be powered by a battery 602. The battery 602 can have 9 volts, though any number of volts can be provided in other variations. The battery 602 can also be rechargeable by any suitable recharging mechanisms. The battery 602 can provide voltage to a power regulator 702 and a stepper motor controller 704. The power regulator 702 can maintain the voltage of the battery 602, for example, reducing voltage to 5 volts. However, the power regulator 702 can reduce the voltage from the battery 602 to any number of volts in other variations. The power regulator 702 can provide the regulated voltage to a microcontroller 706. The microcontroller 706 can be communicatively coupled to or in wireless communication with a Bluetooth radio 708 and the stepper motor controller 704. The microcontroller 706 can be communicatively coupled to or in wireless communication with various peripherals of the device 100, including the Bluetooth radio 708. The stepper motor controller 704 can receive data, commands, or instructions from the microcontroller 706 to power the motor 700 and operate the device. The Bluetooth radio 708 can be in wireless communication with a remote device 800, such as a smartphone device. With this system, programs for controller the stepper motor controller 704 and the motor 700 can be managed via the remote device 800. The compression device 100 can be configured to be in wireless communication with the remote device 800. In the event another compressive mechanism is used to achieve compressive force on the abdomen, the stepper motor controller 704 can be exchanged for any other hardware to achieve the desired compression. That unspecified hardware replacing 704 can be manipulated by micro-controller 706. This hardware can be a pneumatic actuator, a shape-memory wire, or any other hardware. This additional hardware can be powered by micro-controller 706, battery 602, or any other source.

FIG. 8 illustrates various databases communicating with a variation of the compression device 100. The systems can comprise a doctor dashboard 802, a remote device 800, a web database 804, and a mobile database 806 can be provided to control and monitor the device 100. The doctor dashboard 802 can use an algorithm to provide specific programs of care to a subject based on the subject's needs. The doctor dashboard 802 can send information and data to the web database 804. Web database 804 can be a SQL database, though other databases (e.g., no-SQL database or other types of relational databases) can be considered. The web database 804 and the remote device 800 can be in a constant feedback loop such that the remote device 800 retrieves programs from the web database 804 while sending the web database 804 information on specific subject results from the specific programs that have been run. The remote device 800 can also maintain a constant feedback loop with an internal mobile database 806 in the event that wireless connection is lost. The compression device 100 can be configured to apply compressive forces to the abdominal wall of the subject automatically in response to a user input applied to the remote device 800.

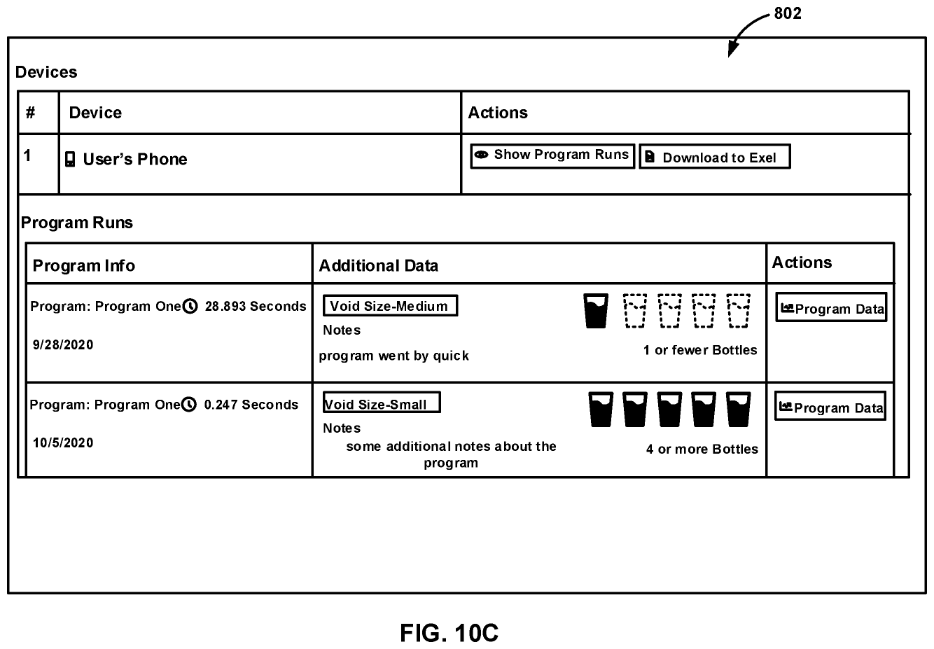

FIGS. 9A to 9D illustrate various graphical user interfaces on display of a variation of the remote device 800. The user interface 900 shown on FIG. 9A illustrates a home screen where the user can check the connection, name, and address of their compression device 100 to be connected to Bluetooth. This screen can allow the user to connect their remote device 800 with the compression device 100 also show how many paired and unpaired devices there are to the remote device 800. From this page, a user can navigate to other functions of the application.

For example, FIG. 9B shows a list of programs that can be accessed by the user. Each program can be designed to trigger a variable amount of pressure at a variable rate for a variable amount of time. The programs can be set by the user or the doctor via the doctor dashboard 802. The programs can be run at any time and can be customized for any particular subject at any particular time. For example, the programs can instruct the compression device 100 to start compression at a certain bladder volume. The programs can vary with respect to parameters such as magnitude of force, compression rate, duration of therapy, etc. Once a program is run or executed, the executed program can be stored in the remote device 800 and the web database 804 for analysis. Information to be stored can comprise: who ran the exercise, the start and stop times of the exercise, first sensation time, first desire time, strong desire time, any changes in sensation over time, void size, fluid intake amount (i.e., how much fluid the subject drank), and additional notes by the subject.

As previously discussed, a user can apply a user input to one of the graphical user interfaces to control the compression device 100. For example, a user can apply a user input (e.g., a touch input) to a sensation meter graphic (e.g., by sliding or otherwise manipulating the sensation meter graphic). The sensation meter graphic can be shown on a display of the remote device 800.

For example, FIG. 9C illustrates another graphical user interface with the slidable sensation meter shown on the display. The slidable sensation meter can track the volume of the bladder. As such, a user can estimate when a particular program will start based on the volume in their bladder. This screen can also indicate to the user whether a particular program is on or off. A time counter can be shown such that the user knows how long the program has been in operation. The screen can show timings of different bladder sensations and desires as recorded by the compression device 100.

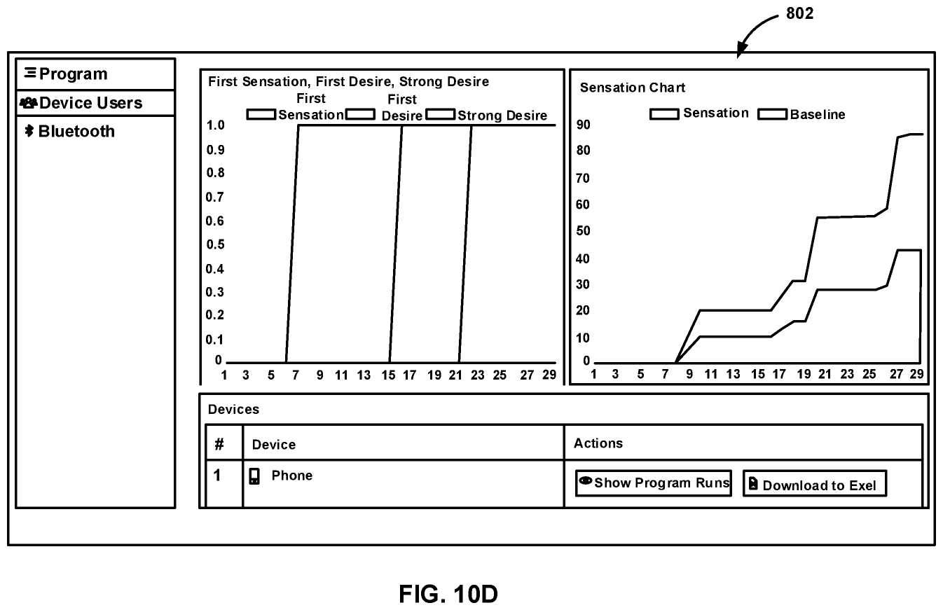

The user interface in FIG. 9D can allow the user to select a fluid intake and a void size amount (i.e., urination volume) after a program or exercise regimen is completed. The user interface can then bring the user to the programs screen as seen in FIG. 9B. The user input is also reported to the doctor dashboard 802. The void size can have levels for the user to select from. For example, the small void size can be anywhere from about 0 mL to about 350 mL, a medium void size can be from about 350 mL to about 700 mL, and a large void size can be from about 700 mL and up. It should be understood that the void size settings can be customizable based on physical characteristics, fluid intake, and other various parameters of an individual subject. Moreover, data and information concerning the subject's fluid intake can be displayed through the doctor dashboard 802 (see, e.g., FIG. 10C).

FIGS. 10A-10D illustrate various displays of a variation of the doctor dashboard 802. The doctor dashboard 802 can offer specific care to individualized patients by monitoring symptoms as they relate to the prescribed therapy. As seen in FIG. 10A, the doctor dashboard 802 can comprise a user interface for a doctor or physician to enter programs specific to a subject. The programs can then be sent to web database 804 and then to the remote device 800 of the subject. From this interface, the user can add or delete programs to be used by a subject, edit the programs, and view details of the program. Changes to programs can be executed at any time via the doctor dashboard 802, making programs dynamic without having to access a subject's remote device.

FIG. 10B illustrates a program page of the doctor dashboard 802. From this interface, the user can view details such as the program name, a description of the program, sensation levels or thresholds that can trigger certain exercises, and what specific exercises are triggered when the sensation meter 902 reaches a predetermined level or threshold level. Tables, graphs, and charts can be automatically generated by the compression device 100 and can enhance patient education and symptom tracking. The user can see details such as a rotational direction (clockwise or counterclockwise) of the threaded shaft, the time of each rotational direction, a degree of compression, and the number of exercise cycles. These parameters can also be customizable via the doctor dashboard 802 and can be created and adjusted without input from the remote device 800 of the subject if necessary.

Once the exercises have started to operate, the user can view certain statistics on the doctor dashboard 802, as seen in FIG. 10D. From this interface, the user can determine at what time a sensation or a desire occurred within the bladder. The user can also graph the difference between a sensation level (i.e., the volume within the bladder) and a predetermined baseline. The comparison can tell a user information about the subject, allowing them to adjust the exercise programs accordingly. The exercise programs can be manually or automatically adjusted in response to feedback from the compression device 100.

The remote device can be configured to provide feedback to the compression device in response to a characteristic of the bladder of the subject. Thus, the compression device 100 can not only exercise the detrusor muscle in the manner as described above, but it can also monitor the bladder environment and the biomechanics of the bladder (e.g., micromotion of the bladder wall, wall tension, bladder volume, etc.). Furthermore, these various forms of feedback can directly control the device and its ability to activate or deactivate.

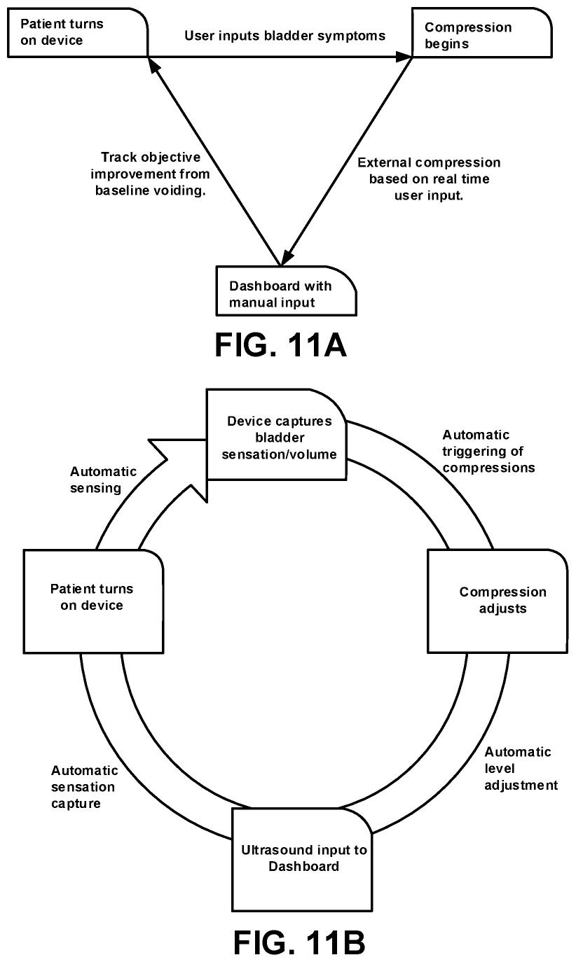

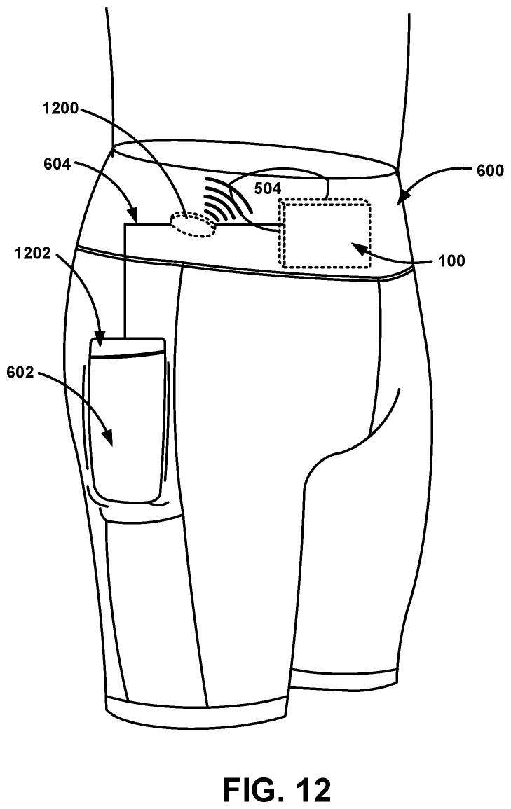

FIGS. 11A-11B illustrate manual and automatic feedback loops of a variation of the compression device 100 and the remote device 800. As such, the compression device 100 can be used both manually and automatically. First, as seen in FIG. 11A, the subject manually turns on the compression device 100. The subject can input bladder symptoms via the remote device 800 in communication with the compression device 100 as described herein. Once the bladder symptoms are input, the compression device 100 can begin to apply compressive forces to the abdominal wall. These initial compressive forces can be based on real-time user input. For example, a user can change the compressive forces based on the comfort level of the subject experienced during compression. The user can then manually input information on the user interface. For example, the user can make notes regarding their sensation levels at certain times. The device can then track their sensation levels to a certain bladder volume using the ultrasound probe. This provides a baseline sensation volume that can be used to calibrate future programs for the particular subject. Eventually, the remote device can collect manual user inputs to track an objective improvement of sensation from a baseline voiding level. The device can adjust certain variables when the remote device 800 communications with the microcontroller 706.

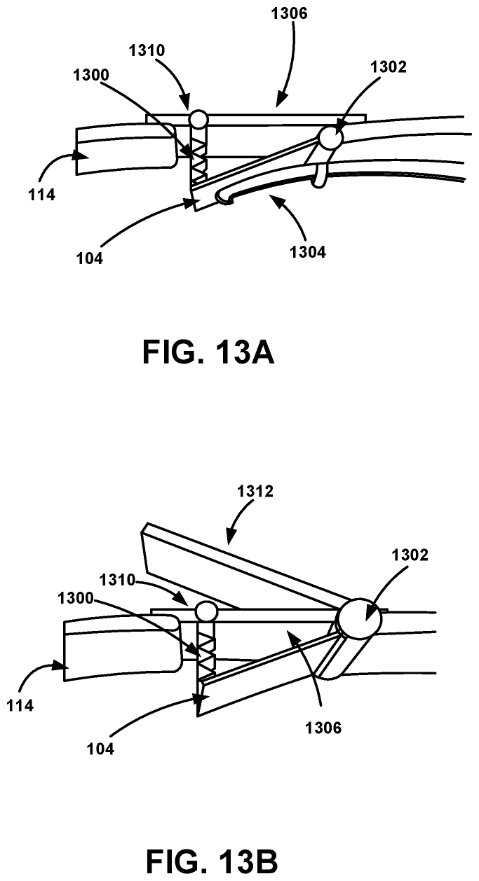

From this point on, the system can automatically operate, as seen in FIG. 11B. As such, the user again can turn on the device to begin sensing the volume within the bladder. The ultrasound probe (as seen in FIG. 12) within the compression device 100 can automatically sense the volume within the bladder of the subject. Accordingly, the compression device 100 can capture the bladder volume and thus the sensation level within the subject. The compression device 100 then can select to trigger specific programs from either the web database 804 or the mobile database 806, applying compressions of the abdominal wall based on data gathered from previous cycles of compressions and/or user input into the diary function of the user interface. The compression device 100 can be adjusted by information including, but not limited to rate, time, magnitude, rotation, type of compression, vibration, intensity of the heat, or other parameters. As such, all of the parameters and information within the system can be individualized for the specific subject.

During this process, the ultrasound probe can be constantly gathering data regarding the volume, shape, or motion within the bladder. The ultrasound probe can be configured to provide an input to the remote device 800, leading to information regarding the sensation levels of the subject. This capturing of sensation levels can occur automatically and continues the automatic feedback loop such that the user does not need to manually input until they desire to. The compressions can also adjust according to previous cycles of compressions. With all of these components, compression device 100 can employ cross-sectional analysis to determine elastic properties of the bladder and combine this information with bladder volume or shape change for optimally timed and designed bladder compressions.

FIG. 12 illustrates a garment having a variation of the compression device and a battery coupled thereto. As also described in FIG. 6, the shape of the compression device can be such that a subject can wear the device discreetly. To power the compression device, a battery 602 can be provided to be held within the garment 600.

The compression device 100 can further comprise an ultrasound unit 1202 comprising an ultrasound probe. The ultrasound unit can be configured to detect a volume within the bladder of the subject. The compression device 100 can be communicatively coupled to the ultrasound unit. The compression device 100 can be configured to automatically apply compressive forces to the abdominal wall of the subject in response to the volume detected by the ultrasound unit 1202.

The ultrasound probe 1200 can be held within the belt 114, though it should be understood that the ultrasound probe 1200 can be incorporated into the compression device 100 itself or configured as a standalone element. Additionally, ultrasound sensors can also be built into the belt 114 (e.g., along a length of the belt 114). The ultrasound probe 1200 can be positioned in such a manner such that ultrasound sensors within the probe can have proper contact with the bladder of the subject. The ultrasound probe 1200 can be configured as a non-invasive sensor and can constantly monitor the bladder 504 of the subject. Alternatively, the ultrasound probe 1200 can be secured directly to the subject via medical tape or other suitable attachment mechanisms.

An ultrasound unit 1202 can be embedded into the battery 602 as seen in FIG. 12. Alternatively, the ultrasound unit 1202 can be a standalone unit. The battery 602 can be shaped and configured to sit within the garment 600 as discreetly as desired. The ultrasound unit 1202 can communicate with the ultrasound probe 1200 to collect data gathered from the volume of the bladder when the bladder is contracting, expanding, or in a resting state. The data can track changes in bladder volume, record urination timings, and can send alerts to the remote device 800. then be used in a feedback loop to manually or automatically adjust compressive forces to the abdominal wall as described above.

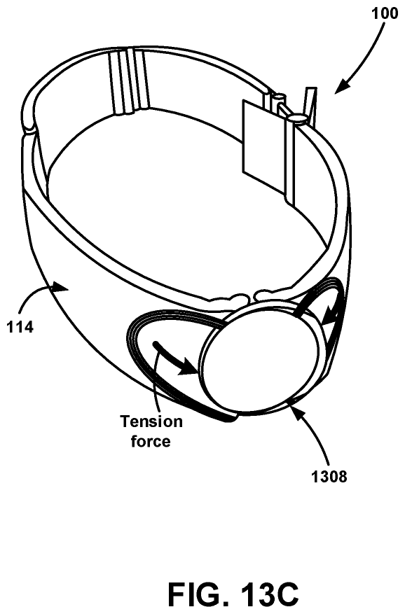

FIGS. 13A-13C illustrate belts 114 having different tensioning mechanisms. For example, FIG. 13A illustrates a mechanical ratchet mechanism 1300 comprising a ratchet hinge 1302 configured to extend and/or expand the device housing 104 placed over the abdomen. The ratchet mechanism 1300 can comprise a tension line 1304 that rotates the device housing 104 of the compression device 100 to apply pressure to the abdomen. Tension can be generated using a lever or wheel to increase and decrease tension via a tension line 1304. The ratchet mechanism 1300 can hold the part in place to sustain the pressure. The ratchet mechanism 1300 can be released via release mechanism 1300 by rotating it. A rigid plate 1306 can be used to support the unit, applying force directed inward towards the abdomen.

As seen in FIG. 13B, other iterations can replace the tensioner and tension line with a lever 1312 that can directly apply force to the device housing 104. A ratchet hinge 1302 can rotate the device housing 104 as the lever 1312 is rotated. Releasing the ratchet mechanism 1300 resets the compression device 100 to its original position.

FIG. 13C shows that the tensioner 1308 can be used to adjust the belt 114 to a tightness level appropriate for the device to function properly. The tensioner 1308 can be positioned near the device at the front of the abdomen or at the side of the belt as shown below, or at the rear of the belt. The user can also adjust the overall circumference of the strap by placing the prong of the buckle into one of the various holes placed into the strap in a linear fashion.

Each of the individual variations or embodiments described and illustrated herein has discrete components and features which may be readily separated from or combined with the features of any of the other variations or embodiments. Modifications may be made to adapt a particular situation, material, composition of matter, process, process act(s) or step(s) to the objective(s), spirit or scope of the present disclosure.

Methods recited herein may be carried out in any order of the recited events that is logically possible, as well as the recited order of events. Moreover, additional steps or operations may be provided or steps or operations may be eliminated to achieve the desired result.

Furthermore, where a range of values is provided, every intervening value between the upper and lower limit of that range and any other stated or intervening value in that stated range is encompassed within the disclosure. Also, any optional feature of the inventive variations described may be set forth and claimed independently, or in combination with any one or more of the features described herein. For example, a description of a range from 1 to 5 should be considered to have disclosed subranges such as from 1 to 3, from 1 to 4, from 2 to 4, from 2 to 5, from 3 to 5, etc. as well as individual numbers within that range, for example 1.5, 2.5, etc. and any whole or partial increments there between.

All existing subject matter mentioned herein (e.g., publications, patents, patent applications) is incorporated by reference herein in its entirety except insofar as the subject matter may conflict with that of the present disclosure (in which case what is present herein shall prevail). The referenced items are provided solely for their disclosure prior to the filing date of the present application. Nothing herein is to be construed as an admission that the present disclosure is not entitled to antedate such material by virtue of prior disclosure(s).

Reference to a singular item, includes the possibility that there are plural of the same items present. More specifically, as used herein and in the appended claims, the singular forms “a,” “an,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise, all technical and scientific terms used herein have the same meaning as commonly understood by one of ordinary skill in the art.

In understanding the scope of the present disclosure, the term “comprising” and its derivatives, as used herein, are intended to be open-ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Also, the terms “part,” “section,” “portion,” “member” “element,” or “component” when used in the singular can have the dual meaning of a single part or a plurality of parts. As used herein, the following directional terms “forward, rearward, above, downward, vertical, horizontal, below, transverse, laterally, and vertically” as well as any other similar directional terms refer to those positions of a device or piece of equipment or those directions of the device or piece of equipment being translated or moved. Finally, terms of degree such as “substantially”, “about” and “approximately” as used herein mean a reasonable amount of deviation (e.g., a deviation of up to ±0.1%, ±1%, ±5%, or ±10%, as such variations are appropriate) from the specified value such that the end result is not significantly or materially changed.

This disclosure is not intended to be limited to the scope of the particular forms set forth, but is intended to cover alternatives, modifications, and equivalents of the variations or embodiments described herein. Further, the scope of the disclosure fully encompasses other variations or embodiments that may become obvious to those skilled in the art in view of this disclosure.

What is claimed is:

1. A bladder control apparatus, comprising:
a garment or accessory configured to be worn at least partially around a lower truncal region of a subject; and
a compression device coupled to the garment or accessory and configured to pressurize an abdominal wall of the subject when the garment or accessory is worn by the subject, the compression device comprising:
a device housing comprising a contact surface,
a device base,
a threaded shaft coupling the device base to the device housing,
an actuator configured to translate the device housing in relation to the device base in a medial or lateral direction relative to the subject such that the contact surface of the device housing applies compressive forces to the abdominal wall of the subject when translated, and
a portable power supply configured to power the actuator.

2. The bladder control apparatus of claim 1, wherein the actuator comprises a shape memory actuator configured to translate the device housing in relation to the device base in response to a temperature change.

3. The bladder control apparatus of claim 2, further comprising a heating element, wherein the heating element is configured to apply heat to the shape memory actuator to change a shape of the shape memory actuator.

4. The bladder control apparatus of claim 3, wherein the shape memory actuator comprises one or more shape memory flaps configured to translate the device housing in relation to the device base in response to the temperature change.

5. The bladder control apparatus of claim 3, wherein the shape memory actuator comprises one or more shape memory springs configured to translate the device housing in relation to the device base in response to a temperature change.

6. The bladder control apparatus of claim 5, wherein the one or more shape memory springs are configured to change a shape of a deformable sheet making up part of the device housing.

7. The bladder control apparatus of claim 2, wherein the shape memory actuator is made in part of at least one of a shape memory alloy and a shape memory polymer.

8. The bladder control apparatus of claim 1, wherein the actuator comprises:
a threaded shaft coupling the device base to the device housing; and
a rotary gear configured to rotate the threaded shaft to translate the device housing in relation to the device base.

9. The bladder control apparatus of claim 1, wherein the compression device further comprises a heating element configured to apply heat to the abdominal wall of the subject.

10. The bladder control apparatus of claim 1, wherein the compression device is configured to be in wireless communication with a remote device, and wherein the compression device is configured to apply compressive forces to the abdominal wall of the subject automatically in response to a user input applied to the remote device.

11. The bladder control apparatus of claim 10, wherein the user input is a manipulation of a sensation meter graphic shown on a display of the remote device.

12. The bladder control apparatus of claim 1, wherein the compression device further comprises an ultrasound unit comprising an ultrasound probe, wherein the ultrasound unit is configured to detect a volume within a bladder of the subject.

13. The bladder control apparatus of claim 12, wherein the compression device is communicatively coupled to the ultrasound unit, wherein the compression device is configured to automatically apply compressive forces to the abdominal wall of the subject in response to the volume detected by the ultrasound unit.

14. The bladder control apparatus of claim 1, wherein the contact surface is substantially planar.

15. The bladder control apparatus of claim 1, further comprising a fluid-filled covering configured to cover at least part of the device housing and wherein the fluid-filled covering is configured to conform to an anatomy of the subject.

16. The bladder control apparatus of claim 15, wherein the fluid-filled covering is filled with air or another type of gas.

17. The bladder control apparatus of claim 15, wherein the fluid-filled covering is filled with a liquid fluid.

18. The bladder control apparatus of claim 15, wherein the fluid-filled covering is filled with particulates.

19. A method of controlling a bladder of a subject, the method comprising:
securing a compression device to the subject with a garment or accessory configured to be worn at least partially around a lower truncal region of a subject, the compression device coupled to the garment or accessory and configured to pressurize an abdominal wall of the subject when the garment or accessory is worn by the subject, the compression device comprising:
a device housing comprising a contact surface,
a device base,
a threaded shaft coupling the device base to the device housing,
an actuator configured to translate the device housing in relation to the device base in a medial or lateral direction relative to the subject such that the contact surface of the device housing applies compressive forces to the abdominal wall of the subject when translated, and
a portable power supply configured to power the actuator; and
translating the device housing in relation to the device base such that the contact surface of the device housing applies compressive forces to the abdominal wall of the subject when translated.

20. A bladder control system, comprising:
a garment or accessory configured to be worn at least partially around a lower truncal region of a subject; and
a compression device coupled to the garment or accessory and configured to pressurize an abdominal wall of the subject when the garment or accessory is worn by the subject, the compression device comprising:
a device housing comprising a contact surface,
a device base,
a threaded shaft coupling the device base to the device housing,
an actuator configured to translate the device housing in relation to the device base in a medial or lateral direction relative to the subject such that the contact surface of the device housing applies compressive forces to the abdominal wall of the subject when translated, and
a portable power supply configured to power the actuator; and
a remote device in wireless communication with the compression device, wherein the remote device is configured to provide feedback to the compression device in response to characteristics of a bladder of the subject.
